(19) United States
(12) Patent Application Publication
(10) Pub. No.: US 2005/0142215 A1
(43) Pub. Date: Jun. 30, 2005

(54) Antimicrobial compositions and methods of use

(76) Inventor: William O. Kling, Dallas, TX (US)

Correspondence Address: Lekha Gopalakrishnan, Esq.;Jenkens & Gilchrist, P.C., 1445 Ross Avenue, Suite 3200, Dallas, TX, 75202-2799, US

(21) Appl. No.: 10/394,619

(22) Filed: Mar. 26, 2003

Related U.S. Application Data
(60) Provisional application No. 60/367,915, filed on Mar. 27, 2002.

(51) Int. Cl. A61K 33/14

(52) U.S. Cl. 424/661

(57) ABSTRACT

The present invention relates to antimicrobial compositions useful in the treatment of microbial and mycotic infections. The antimicrobial compositions are aqueous-based and preferably contain an oxidizing agent as an antimicrobial agent. In certain embodiments of the invention, the antimicrobial agent is chlorine dioxide.

### CROSS REFERENCES TO RELATED APPLICATIONS

This application claims priority to U.S. Provisional Application Ser. No. 60/367,915 filed on Mar. 27, 2002, which is fully incorporated by reference herein.

### BACKGROUND OF THE INVENTION

1. Technical Field of the Invention

The present invention relates to antimicrobial compositions useful in the treatment of microbial infections. The present invention also relates to compositions that are useful for topical application, especially to nails and adjacent tissue for the treatment of mycotic and microbial infections.

2. Description of the Prior Art

Fungal infections of nails, both toe nails and finger nails, are a widespread problem, especially to people with compromised peripheral circulation such as the elderly, chronically ill, and diabetic. Others afflicted with such infections include workers in the medical field, farmers, persons with military service backgrounds, and users of acrylic nail care products. These infections cause irritation and are not easily eliminated, even with repeated applications of commonly prescribed treatments.

Biologically active antimicrobial compounds have proved difficult to administer by the topical route for the treatment of mycotic (yeast, mold, and fungal) and bacterial infections of human nails and adjacent tissue. Commonly applied topical formulations such as creams, ointments, tinctures, aqueous and non-aqueous solutions and suspensions and the like are typically absorbed or rubbed off onto clothing when applied to nails and adjacent tissue; likewise, formulations applied topically to the fingernails and adjacent tissue are typically absorbed onto clothing or gloves or rubbed off incidently during hand washing.

Moreover, it has not been adequately demonstrated that the previously used topical formulations deliver therapeutically adequate doses of the biologically active antimicrobial material to the mycotic or bacterially infected tissue when applied to the nail and adjacent tissue. One reason is the target microbes cause dead skin and/or nail tissue to build up under the nail, blocking access to the microbes by the antimicrobial material.

Various attempts have been made to address the inadequacies of the topical administration of the antimicrobial compositions including the development of various medicating devices for the human nails and adjacent tissues. An example of such a device is set forth in U.S. Pat. No. 5,181,914 which discloses a device which occlusively covers the targeted tissue area with a bandage type device having a medication reservoir. The therapeutic efficacy of such devices markedly depends on the specific biologically active compound employed and the ability of the applied formulation to effectively deliver the active compound to the affected tissue. Furthermore, such treatment requires that the patient wear such a device throughout the treatment.

Research relating to the treatment of infections has primarily centered around the compositions used for treating the infected tissue areas. Various compositions have been developed and used by the medical industry. For example, a composition containing the water insoluble fungistatic agent undecylenic acid with the topical antiseptic agent chloroxylenol in an oil based solvent has been marketed. Additionally, a composition containing the fungistatic agent triacetin, topical antiseptic agents chloroxylenol and cetyl pyridinium chloride, alcohols, and a keratolytic agent glacial acetic acid in an aqueous tincture with several cosmetic preservatives has been marketed for the treatment of infected tissue areas. The disadvantage of the aforementioned compositions is that they contain acids, alcohols and other non-aqueous solvents which can cause contact dermatitis if topical administration is chronic or in sufficiently high concentration. Furthermore, several of the compositions may be ineffective in delivering the active agent to the human nails and adjacent tissue and are thus of unproven therapeutic value. In addition, these compositions are not able to penetrate and access the area under the nail and thus cannot be used for removing debris from under the nail

There is therefore a need to develop an antimicrobial composition which can deliver the antimicrobial agent effectively to the site of the infection, such as on or under an infected nail and in surrounding tissue area. The antimicrobial compositions should preferably contain an effective antimicrobial agent, and be in a form that can effectively deliver that active agent to the infected area when the composition is topically administered to the affected area, preferably by irrigating the nail and surrounding tissue.

### SUMMARY OF THE INVENTION

The present invention provides antimicrobial compositions comprising a biologically effective, therapeutic, non-toxic quantity of an oxidizing agent that functions as an antimicrobial agent. In certain embodiments of the invention, an effective amount of chlorite ion serves as an oxidizing agent and antimicrobial agent. In certain embodiments of the invention, chlorine dioxide or chlorine dioxide generating compounds may be used as the oxidizing agent and antimicrobial agent.

In some embodiments of the invention, the antimicrobial agent may be present in the composition in an admixture with a surfactant. In other embodiments of the antimicrobial composition of the invention, one or more microbicides or fungicides may be used in combination with, chlorine dioxide or chlorine dioxide generating compounds.

The antimicrobial compositions of the present invention are useful in reducing the extent of a microbial infection on the body of an animal, such as a human. The compositions are administered topically, i.e, on the surface, either cutaneously (on the surface of the skin or nail) or to the region under the nail. The administration is preferably to the skin, hair, or nails and surrounding tissue of an animal or human. The preferred application of the compositions is for the treatment of mycotic and bacterially infected human nails and adjacent tissue. In addition to the treatment of microbial and mycotic infections, the antimicrobial compositions of the present invention also serve to rid the treatment area of cellular debris that accumulates as a result of the infection.

### DETAILED DESCRIPTION OF ILLUSTRATIVE EMBODIMENTS

The present invention provides antimicrobial pharmaceutical compositions having oxidizing properties, and methods for their application to cutaneous and subcutaneous tissue and also mucous membrane tissues. The present invention also provides methods of preparing these antimicrobial pharmaceutical compositions. The antimicrobial compositions of the present invention comprise at least one biologically active antimicrobial agent. In certain embodiments of the invention, the biologically active antimicrobial agent possesses oxidizing properties.

In certain embodiments of the invention, the antimicrobial composition further comprises one or more surfactants.

The antimicrobial composition of the present invention comprises at least one antimicrobial agent at a biologically effective, therapeutic, non-toxic concentration. Unless explicity stated otherwise, all weight percentages in the specification and claims are based on the total weight of the antimicrobial composition.

The antimicrobial agents present in the antimicrobial compositions are preferably chlorine dioxide generating compounds. Examples of such chlorine dioxide generating compounds include, but are not limited to, sodium chlorite, sodium chlorate and chlorite ion. The terms “chlorine dioxide generating compound” and “chlorine dioxide compound” are used interchangeably herein. In an embodiment of the invention, the chlorine dioxide compound is an aqueous solution comprising chlorine dioxide. The aqueous solution is prepared by dissolving chlorine dioxide gas in purified water.

In certain embodiments of the invention, the antimicrobial compositions of the present invention may further comprise one or more surfactants. Surfactants are used to atabilize the chlorine dioxide compound in an aqueous solution. The surfactants include, but are not limited to, sodium monooleate, lauryl polyglucose and cocoamphodiacetate. In an embodiment of the invention, the surfactant used is a non-ionic surfactant. The surfactant can be present in the range of from about 0.0001 to about 8 wt %. In an alternate embodiment, the surfactant is present at a range of from about 0.001 wt % to about 5 wt %.

Chlorine dioxide is a very strong oxidant and exerts its microbicidal properties through an oxidizing process. The chlorine dioxide compound of the present invention performs its antimicrobial function by carrying out an oxidation process at the point of application. In an embodiment of the invention, the chlorine dioxide compound of the present invention oxidizes the area of topical application. This oxidation process results in the cessation of the microbial or mycotic infection by virtue of the killing of the causative microbe or fungus. The oxidation process also results in the removal of cellular debris comprised of largely dead skin and tissue.

The antimicrobial compositions of the present invention are aqueous-based solutions. Preferably, the aqueous solvent is purified water in accordance with the USP standard for purified water. The amount of water in the compositions is generally at least about 60 wt %, i.e., the amount of water in the composition is present at a concentration of not less than 60 wt % of the composition. In preferred embodiments of the invention, the concentration of water is preferably at least about 70 wt %, more preferably at least about 80 wt %, and even more preferably at least about 85 wt %.

The antimicrobial compositions of the present invention can optionally include other compounds including, but not limited to, sodium bicarbonate, sodium hydroxide, triethanolamine, citric acid and lactic acid, which are used to adjust/buffer the pH of the composition. Other components that may be optionally included in the antimicrobial compositions of the present invention include disodium ethylene diamine tetraacetic acid (EDTA) as a chelating agent, and hydroxyethylcellulose or carbomer as a thickener to achieve viscosities within a useful range appropriate for the mode of application.

An embodiment of the invention provides an antimicrobial composition having a pH ranging from about 6 to about 11. In an preferred embodiment, the pH of the composition ranges from about 7 to about 9.

The concentration of the chlorine dioxide compound present in the antimicrobial compositions ranges from about 0.005 to about 0.5 wt %. In an alternate embodiment of the invention, the concentration of the chlorine dioxide composition ranges from about 0.01 to about 0.4 wt %. In yet another embodiment of the invention, the concentration of the chlorine dioxide compound ranges from about 0.03 wt % to 0.3 wt %.

The antimicrobial compositions of the invention are effective in killing and altering bacterial structure and metabolism as well as suppressing the growth of microorganisms that cause infection in and around human nails and surrounding tissue. In an embodiment of the invention, the antimicrobial composition is topically administered to an infected area requiring treatment. Examples of typical areas requiring treatment include skin, hair and nails of animals and humans.

In other embodiments of the antimicrobial composition of the invention, one or more microbicides or fungicides may be used in combination with, chlorine dioxide or chlorine dioxide generating compounds. The microbicides are selected from the group consisting of benzalkonium chloride, benzethonium chloride, hexylresorcinol, hydrogen perioxide solution tincture of iodine, iodine topical solution, isopropyl alcohol, methylbenzethonium chloride and phenol. In a preferred embodiment of the invention, the microbicide used in combination with the chlorine dioxide compound of the present invention is benzalkonium chloride.

An embodiment of the invention provides an antimicrobial composition for the treatment of nail fungus. Nail fungus is caused by common fungi which are present in the environment. These fungi flourish in the warm and moist environment present under the nail. Reportedly, a great majority of the nail fungus is caused by Tricophyton mentagrophytes and Tricophyton rubrum. As the fungus grows, dead tissue builds up under the nail and on top of the nail. The nail begins to grow in a distorted form and eventually disintegrates due to fungus growth. The fungus growing under the nail cannot be killed easily because of its location. In addition, it becomes more difficult to access and kill the fungus after cellular debris has accumulated on the nail, because this further blocks the ability of the antimicrobial agent to flow under the nail and reach the fungus. In order to combat nail fungus and related maladies, the antimicrobial composition of the present invention effectively delivers an antimicrobial agent to the site of the infection, such as an infected nail and surrounding tissue area. In addition to fighting the infection in the treated area, the antimicrobial composition also breaks down and degrades any dead cell/dead tissue debris in and around the area being treated.

In certain embodiments, the antimicrobial compositions of the present invention are used to prevent the onset of a microbial infection. Certain embodiments of the invention provide a method for preventing the onset of a microbial infection comprising, administering a composition comprising:
(a) a chlorine dioxide compound present at a concentration ranging from about 0.005 to about 0.5 weight percent of said composition; and,
(b) at least about 60 wt % water.

### WORKING EXAMPLES

Test Organisms:

Cultures of the following microorganisms are maintained as stock cultures from which working inoculum are prepared. The viable microorganisms used in this test must not be more than five passages removed from the original stock culture. For purposes of the test, one passage is defined as the transfer of organisms from an established culture to fresh medium. All transfers are counted.
A. Tricophyton mentagrophytes (ATCC No. 9533)
B. Tricophyton rubrum (ATCC No. 28188)
   Materials:
A. Test tubes with closures
B. Pipettes, 10.0 ml and 1.0 ml serological
C. 0.85% Phosphate buffered saline or peptone water, pH 7.0-7.2
D. Petri dishes, culture loops, and other microbiological apparatus
   Media:
A. Tryptic Soy Agar with lecithin and tween 80
B. Sabouraud Dextrose Agar or Potato Dextrose Agar
   Procedure:
A. Preparation of Test Samples:
   1. Accurately pipette 9.9 ml of product into each of six appropriately labeled or coded test tubes.
   2. Store test samples at ambient temperature.
B. Preparation of Inoculum:
1. Inoculate the surface of a suitable volume of solid agar medium from a recently grown stock culture of each of the specified microorganisms. Incubate the T. rubrum and T. mentagrophytes cultures for one week.
2. To harvest the T. rubrum and T. mentagrophytes cultures, remove the cultures by adding sterile saline to the plate/slant and scraping the surface of the culture with a pipette or loop. Alternatively, glass beads may be used for slant cultures. Adjust the count with sterile saline so that the concentration of the inoculum level is between 100,000 and 1,000,000 microorganisms per ml or gram of product.
3. Determine the number of viable microorganisms in each milliliter of the inoculum suspensions by serial dilution in sterile phosphate buffered saline:
4. Plate dilutions of 10⁻², 10⁻⁴, 10⁻⁶, and 10⁻⁷ for all organisms.
5. Overlay with approximately 20 ml of 45° C. Tryptic Soy Agar with lecithin and Tween 80 or Sabouraud Dextrose Agar depending on microorganism being cultured.
6. Incubate for 48 hours at 30-35° C. for all test organisms. Incubate T. rubrum and T. mentagrophytes an additional 48 hours at 20-25° C.
7. Count test organisms.
8. Calculate the number of organisms as colony forming units per ml (cfu/ml) of inoculum as follows:

$$\frac{cfu \text{/} ml ( 0.1 ml )}{9.9 ml} = cfu \text{/} ml of product$$
C. Inoculation and Plating of Samples:
   1. Aseptically transfer 0.1 ml of each test suspension into the appropriately labeled 9.9 ml sample of test material containing 0.09 wt % of chlorine dioxide compound. Each test organism is inoculated as a pure culture into a single 9.9 ml sample of test material.
   2. Thoroughly mix or stir all samples by vortex.
   3. Let stand for 5 minutes and 30 minutes. Vortex sample every minute for the 5 minutes, and every 5 minutes for 30 minutes.
   4. Remove aliquots at 5 minutes and 30 minutes and transfer to 9.9 ml sterile saline.
   5. Perform serial dilutions from 10⁻² to 10⁻⁶
   6. Transfer 1.0 ml of each dilution into a 100×15 mm petri plate in duplicate.
   7. Overlay with approximately 20 ml of 45° C. Tryptic Soy Agar with lecithin and Tween 80 or Sabouraud Dextrose Agar depending on microorganism being cultured.
   8. Gently swirl plates and allow to solidify.
   9. Incubate plates for 48 hours at 35° C. and 48 hours at 25° C.
D. Sample Evaluation:
   1. Read plates and record results on appropriate data sheet.
   2. Using the calculated inoculum concentration of each test microorganism, calculate the log reduction of each microorganism for each kill rate.
      Data:

A. Kill rate Results

TABLE 1
5-minute Results

| | T. menta ATCC 9533 | T. rubrum ATCC 28188 |
|---|---|---|
| Inoculum level | 2.83 × 10⁵ | 1.16 × 10⁴ |
| Direct | Too numerous to count | Too numerous to count |
| 10⁻² | 20 | 23 |
| 10⁻⁴ | 0 | 0 |
| 10⁻⁵ | 0 | 0 |
| Average Count | 2.15 × 10³ | 0 |
| Log Reduction | 2 | 4 |

TABLE 2
30-Minute Results

| | T. menta ATCC 9533 | T. rubrum ATCC 28188 |
|---|---|---|
| Inoculum level | 2.83 × 10⁵ | 1.16 × 10⁴ |
| Direct | 0 | 0 |
| 10⁻² | 0 | 0 |
| 10⁻⁴ | 0 | 0 |
| 10⁻⁵ | 0 | 0 |
| Average Count | 0 | 0 |
| Log Reduction | 5 | 4 |

Testing of the Invention:

The compositions of the present invention were evaluated in a study of five human subjects having fungal infections of the nails. The study was designed and conducted by an independent testing laboratory.

All patients exhibited clinically apparent symptoms of onychomycosis in the form of thick, discolored, crumbly and/or dystrophic nails in varying degrees of severity. Test subjects consisted of five adults over the age of forty, three females and two males. One female subject was a diabetic.

Testing of the product consisted of five adults applying the product to appropriate nail(s) and surrounding tissue area. Product was applied so that the liquid was placed under the toenail, on the toenail and surrounding skin. The product was rubbed on the surface of the nail. The product was applied twice daily and allowed to dry prior to placing socks and shoes on the feet.

Short Term Test Results

Following four weeks of chlorine dioxide solution treatment (0.16 wt % chlorine dioxide in an aqueous solution), the following results were documented.
1. All test subjects improved significantly, with an average beginning score of 5.0 for the population and an average 7.6 score on a scale of 1 to 10 after four weeks.
2. Average score improvement was 34% over four weeks, with improvement ranging from 12% to 83%. In general, those with the worst nail condition at the beginning showed the highest percentage improvement over the four week period.
3. The elderly female diabetic subject, who had a beginning score of 1, showed a marked decrease in brittleness and improved clarity of the nails. The powdery effect has significantly subsided and nails no longer flake into a powder when clipped. The base of the nail is growing as a clear nail. This is the first instance of a clear nail growing on the subject in years.

4. All subjects note the nails appear healthier-looking, color and clarity is more natural and nails are significantly easier to clip.

Scoring of Subjects on a Scale of 1 to 10

| | |
|---|---|
| Score of 1: | Toenails discolored to a dark yellow-brown |
| | Toenails are opaque |
| | Toenails are brittle |
| | Toenails flake (powder like substance) upon clipping |
| | Toenails grow unevenly |
| | Toenails are difficult to clip |
| | Toenails or skin may itch or feel irritated |
| Score of 3: | Toenails discolored to a dark yellow-light brown |
| | Toenails are opaque |
| | Toenails are brittle |
| | Toenails flake (powder like substance) upon clipping |
| | Toenails grow unevenly |
| | Toenails are difficult to clip |
| Score of 5: | Toenails discolored to a yellow color |
| | Toenails are opaque |
| | Toenails are brittle |
| | Toenails flake (powder like substance) upon clipping |
| | Toenails may grow unevenly (not all nails affected) |
| | Toenails are difficult to clip |
| Score of 7: | Toenails discolored light yellow or slightly opaque |
| | Toenails may be brittle (not all nails affected) |
| Score of 10: | Toenails appear normal and healthy |
| | No discoloration of nails |
| | Toenails are easy to clip |

TABLE 3

| Subject | Initial Score | Score after 4 weeks |
|---|---|---|
| Male 1 | 5 | 8 |
| Male 2 | 5 | 8 |
| Male 3 | 7 | 8 |
| Female 1 | 7 | 8 |
| Female 2 | 1 | 6 |

1. A method for treatment of an infection, comprising administering a composition comprising:
(a) a chlorine dioxide compound present at a concentration ranging from about 0.005 to about 0.5 weight percent of said composition; and,
(b) at least about 60 weight percent water.

2. The method of claim 1, wherein said composition further comprises one or more surfactants.

3. The method of claim 1, wherein said composition further comprises a chelating agent.

4. The method of claim 1, wherein said composition further comprises a thickener.

5. The method of claim 1, wherein said composition further comprises one or more microbicides.

6. The method of claim 1, wherein said composition is administered to the skin of a human.

7. The method of claim 1, wherein said composition is administered to the nails of a human.

8. The method of claim 1, wherein said chlorine dioxide compound is selected from the group consisting of sodium chlorite and sodium chlorate.

9. The method of claim 1, wherein said chlorine dioxide compound is an aqueous solution comprising chlorine dioxide.

10. The method of claim 1, wherein the concentration of said chlorine dioxide compound ranges from about 0.01 wt % to about 0.4 wt %.

11. The method of claim 1, wherein the concentration of said chlorine dioxide compound ranges from about 0.03 wt % to about 0.3 wt %.

12. The method of claim 1, wherein the composition comprises at least about 70 wt % water.

13. The method of claim 5, wherein said one or more microbicides are selected from the group consisting of benzalkonium chloride, benzethonium chloride, hexylresorcinol, hydrogen peroxide solution, tincture of iodine, iodine topical solution, isopropyl alcohol, methylbenzethonium chloride and phenol.

14. A method for preventing the onset of a microbial infection, comprising administering a composition comprising:
(a) a chlorine dioxide compound present at a concentration ranging from about 0.005 to about 0.5 weight percent of said composition; and,
(b) at least about 60 wt % water.

15. The method of claim 14, wherein said composition further comprises one or more surfactants.

16. The method of claim 14, wherein said composition further comprises a chelating agent.

17. The method of claim 14, wherein said composition further comprises a thickener.

18. The method of claim 14, wherein said composition further comprises one or more microbicides

19. The method of claim 14, wherein said composition is administered to the skin of a human.

20. The method of claim 14, wherein said composition is administered to the nails of a human.

21. The method of claim 14, wherein said chlorine dioxide compound is an aqueous solution comprising chlorine dioxide.

22. The method of claim 14, wherein the concentration of said chlorine dioxide compound ranges from about 0.01 wt % to about 0.4 wt %.

23. The method of claim 14, wherein the concentration of said chlorine dioxide compound ranges from about 0.03 wt % to about 0.3 wt %.

24. A composition for topical administration of an antimicrobial agent comprising
(a) a chlorine dioxide compound present at a concentration ranging from about 0.005 to about 0.5 weight percent of said composition; and,
(b) at least about 60 wt % water.

25. The composition of claim 24, wherein said composition further comprises one or more surfactants.

26. The composition of claim 24, wherein said composition further comprises a chelating agent.

27. The composition of claim 24, wherein said composition further comprises a thickener.

28. The composition of claim 24, wherein said composition further comprises one or more microbicides.

29. The composition of claim 24, wherein said composition is administered to the nails of a human.

30. The composition of claim 24, wherein said chlorine dioxide compound is selected from the group consisting of sodium chlorite and sodium chlorate.

31. The composition of claim 24, wherein said chlorine dioxide compound is an aqueous solution comprising chlorine dioxide

32. The composition of claim 24, wherein the concentration of said chlorine dioxide compound ranges from about 0.01 wt % to about 0.4 wt %.

33. The composition of claim 24, wherein the concentration of said chlorine dioxide compound ranges from about 0.03 wt % to about 0.3 wt %.

34. The composition of claim 24, wherein the composition comprises at least about 70 wt % water.

35. The composition of claim 28, wherein said one or more microbicides are selected from the group consisting of benzalkonium chloride, benzethonium chloride, hexylresorcinol, hydrogen peroxide solution, tincture of iodine, iodine topical solution, isopropyl alcohol, methylbenzethonium chloride and phenol.

36. The composition of claim 24, wherein the pH of the composition ranges from about 6 to about 11.
